(19) United States
(12) Patent Application Publication
(10) Pub. No.: US 2026/0240193 A1
(43) Pub. Date: Aug. 20, 2026

(54) GUMMY PRODUCT COMPRISING AN OILY INGREDIENT

(71) Applicant: DSM IP Assets B.V., Maastricht (NL)

(72) Inventors: Gang SU, Ellicott City, MD (US); Kristin BRANCATO, Doylestown, PA (US); Xiaoxiao CHEN, Millburn, NJ (US); Zhenbo MA, Metuchen, NJ (US); John RICCIARDI, Basking Ridge, NJ (US)

(73) Assignee: DSM IP Assets B.V., Maastricht (NL)

(21) Appl. No.: 19/513,876

(22) PCT Filed: Aug. 14, 2024
(86) PCT No.: PCT/US2024/042249

§ 371 Date: Feb. 24, 2026

Related U.S. Application Data
(60) Provisional application No. 63/532,757, filed on Aug. 15, 2023.

(30) Foreign Application Priority Data
Sep. 19, 2023 (EP) 23198411.3

(51) Int. Cl. A23G 3/36; A23G 3/40; A23G 3/42; A61K 9/00; A61K 31/00; A61K 31/202; A61K 47/36; A61K 47/46

(52) U.S. Cl. CPC A23G 3/364; A23G 3/368; A23G 3/40; A23G 3/42; A61K 9/0056; A61K 31/202; A61K 31/658; A61K 47/36; A61K 47/46

(57) ABSTRACT

The present invention relates to a new formulation, which is a gummy product comprising a high amount of at least one oily ingredient. The gummy product includes an oily ingredient can be any oil/fat. Suitable oils/fats are i.e. any PUFA, a fat-soluble vitamin, any carotenoid, any cannabinoid oil. The disclosed invention also includes a process for producing said gummy product.

### FIELD

The present invention relates to a new formulation for a gummy product comprising at least one oily ingredient.

### BACKGROUND

There are gummy products known in the art. They are mainly used as candies, such as gummy bears, and similar product forms. The distinct texture and mouthfeel of gummy products result from gelling agents. Gelatin is the original gelling agent used and is still typically the gelling agent of choice in commercial gummy products.

Functional gummy products that contain a desirable nutritional, dietary, or other ingredient are known. Such ingredients may be incorporated in the form of an oil. However, the gummy products of the prior art are limited in the amount of the at least one oily ingredient they contain without having a variety of issues. There can be stability issues, taste issues, issues during production, etc.

The goal of the present invention is to provide gummy products comprising a high amount of at least one oily ingredient, which are easily produced, are storage stable, and have no taste or smell issues.

### SUMMARY

Prior art gummies are typically produced using high amounts of gelatin to achieve the desirable texture and mouthfeel indicative of gummy products. The high amount of gelatin limits the amount of desirable functional ingredients that can be incorporated into a stable gummy product that still exhibits the desirable texture and mouthfeel of gelatin-based gummy products. If reducing the amount of gelatin and increasing the amount of oily ingredient, issues with phase-separation, stability, taste, or smell are observed.

Surprisingly, it was found, that when adding at least one emulsifier to the gummy product, a stable gummy product is obtained using relatively low amounts of gelatin, wherein the at least one oily ingredient is distributed homogenously in the gummy product.

Therefore, the present invention relates to a gummy product comprising:
10-30 wt.-%, based on the weight of the gummy product, of at least one oily ingredient, and
0.5-20 wt.-%, based on the total weight of the gummy product, of at least one emulsifier, and
5-35 wt.-%, based on the total weight of the gummy product, of water, and
1-15 wt.-%, based on the total weight of the gummy product, of at least one gelling agent, wherein the gelling agent comprises less than 4 wt.-% gelatin, and
0.1-70 wt.-%, based on the total weight of the gummy product, of at least one auxiliary agent,
and
   wherein the at least one oily ingredient is distributed homogenously in the gummy product.
   All percentages for one embodiment always add up to 100.

### DETAILED DESCRIPTION

The expression “distributed homogenously in the gummy product” means that the concentration of the at least one oily ingredient is more similar all over the gummy product. This means that the at least one oily ingredient is not concentrated at a specific spot of the gummy product.

The gummy product according to the present invention comprises at least one oily ingredient. The oily ingredient can be any oil/fat.

Suitable oils/fats are i.e. any PUFA, a fat-soluble vitamin, any carotenoid, any cannabinoid oil.

Therefore, the present invention relates to a gummy product, wherein the at least one oil/fat is chosen from the group consisting of PUFAs, fat-soluble vitamins, carotenoids and cannabinoid oils.

The embodiments of the present invention can comprise a high amount of PUFA; and these gummy products are storage stable and have a nice taste.

PUFAs (Poly Unsaturated Fatty Acids) are known per se.

Preferred poly-unsaturated fatty acids are those having from 16 to 24 carbon atoms, preferably from 18 to 22 carbon atoms, preferably with 18, 20 or 22 carbon atoms and having multiple unsaturated carbon-carbon double bonds.
Examples of such poly-unsaturated fatty acids are the known n-3 PUFA's. Preferred are for example poly-unsaturated acids such as n-3 PUFA, mainly as eicosapentaenoic acid (EPA), docosapentaenoic acid (DPA), docosahexaenoic acid (DHA), such as (cis-) 5,8,11,14,17-eicosapentanoic acid and/or (cis-) 4,7,10,13,16,19-docosahexaenic acid. Such PUFAs can be sourced from natural sources, such as walnuts, sunflower seeds, flax seeds, flax oil, fish (such as i.e., salmon, mackerel, herring, albacore tuna, and trout), corn oil, soybean oil, safflower oil.
It is also possible to produce PUFA (bio) chemically, for example by fermentation. PUFAs are available commercially from a variety of suppliers in a variety of forms.

PUFAs (especially the n-3 PUFAs) are known to have variety of health benefits against cardiovascular diseases (CVDs) including well-established hypotriglyceridemic and anti-inflammatory effects, healthy infant development, and brain function in older adults. Also, various studies indicate promising antihypertensive, anticancer, antioxidant, antidepression, antiaging, and anti-arthritis effects.

Preferably, the gummy product according to the present invention comprises at least one PUFA chosen from the group consisting of PUFA having from 16 to 24 carbon atoms and having multiple unsaturated carbon-carbon double bonds.

More preferably, the gummy product according to the present invention comprises at least one PUFA chosen from the group consisting of PUFA having from 18 to 22 carbon atoms and having multiple unsaturated carbon-carbon double bonds.

Even more preferably the gummy product according to the present invention comprises at least one PUFA chosen from the group consisting of PUFA having 18, 20 or 22 carbon atoms and having multiple unsaturated carbon-carbon double bonds.

Most preferably, the gummy product according to the present invention comprises at least one PUFA chosen from the group consisting of eicosapentaenoic acid (EPA), docosapentaenoic acid (DPA) and docosahexaenoic acid (DHA).

The gummy product according to the present invention can comprise a fat-soluble vitamin. Fat-soluble vitamins are vitamins A, D, E, and K, as well as derivates thereof.

The gummy product according to the present invention can comprise any carotenoids.

Carotenoids used in the gummy product according to the present invention are α- or β-carotene, 8′-apo-β-carotenal, 8′-apo-β-carotenoic acid esters such as the ethyl ester, canthaxanthin, astaxanthin, astaxanthin esters, lycopene, lutein, zeaxanthin or crocetin and their derivatives.

The gummy product according to the present invention can comprise any cannabinoid oil.

Cannabinoid types used in the gummy product according to the present invention are cannabidiol (CBD), tetrahydrocannabinol (THC), cannabinol (CBN), cannabigerol (CBG), cannabichromene (CBC), cannabicitran (CBT), cannabinodivarin (CBV), cannabiripsol (CBR), hexahydrocannabinol (HHC), delta-9-tetrahydrocannabiphoral (THCP), tet-rahydrocannabivarin (THCV), endocannabinoid (anandamide) and endocannabinoid (2-AG).

Preferred cannabinoids are chosen from the group consisting of cannabidiol, tetrahydrocannabinol, cannabinol, cannabigerol, cannabichromene, cannabicitran, cannabinodivarin, cannabiripsol, hexahydrocannabinol and delta-9-tetrahydrocannabiphoral.

More preferred cannabinoids are chosen from the group consisting of cannabidiol, tetrahydrocannabinol, cannabinol and cannabigerol (most preferred is cannabidiol).

As disclosed above the content of at least one oily ingredient in the gummy product is 10-30 wt.-%, based on the total weight of the gummy product, of at least one oily ingredient.

Preferably, the content of at least one oily ingredient in the gummy product is 12-30 wt.-%, more preferably 12-25 wt.-%, based on the total weight of the gummy product.

It is possible to use the oily ingredient in its oil form or it is also possible to use a formulation of these compounds, which is in a powder form.

The gummy product comprises at least one emulsifier.

The emulsifier can be any commonly used emulsifier.
Usually and preferably, the emulsifier is food grade (or pharma grade).

Suitable emulsifiers are i.e., modified food starch (such as OSA starch), quillaja extract, gum Arabic, modified gum Acacia, polysorbates, polyglycerol esters of fatty acids, lecithin, sucrose esters, sucroglycerides, and lignosulfonates.

Preferred emulsifiers are modified food starch (more preferred OSA starch), quillaja extract, and gum Arabic.

As disclosed above the gummy product comprises 0.5-20 wt.-%, based on the total weight of the gummy product, of at least one emulsifier.

Preferably, the content of at least one emulsifier in the gummy product is 1-8 wt.-%, more preferably 2-5 wt.-%, based on the total weight of the gummy product.

As disclosed above the center of the gummy product comprises 5-35 wt.-%, based on the total weight of the gummy product, of water.

Preferably, the gummy product comprises 5-30 wt.-%, more preferably 8-25 wt.-%, based on the total weight of the gummy product, of water.

As disclosed above the gummy product comprises at least one gelling agent.

Any commonly known gelling agent (or mixture of gelling agents), which are usually used in gummy products, can be used.

Suitable gelling agents are gelatin (from any source), pectin (from any sources), starches (from any sources), carrageenan, gellan gum, agar, or other hydrocolloids (from any sources). Gelatin is present at less than 4 wt.-% based on the total weight of the gummy product, preferably less than 3.5 wt.-%, more preferably less than 3 wt.-%, more preferably less than 2 wt.-%, more preferably less than 1 wt.-%, and most preferably the gelling agent is substantially devoid or devoid of gelatin.

A preferred gelling agent is pectin. Pectin is derived from plant sources. Pectin may be more suitable than gelatin for those having dietary restrictions or ethical or reli-gious objections to the consumption of gelatin. Additionally, pectin may be less likely to cause allergic reactions than gelatin.

In an embodiment, the gelling agent consists of a pectin, a starch, or a mixture thereof.

As disclosed above the gummy product comprises 1-15 wt.-%, based on the total weight of the gummy product, of at least one gelling agent.

Preferably, the gummy product comprises 1-12 wt.-%, more preferably 2-10 wt.-%, more preferably 2-8 wt.-%, more preferably 2-7 wt.-%, more preferably 2-6 wt.-%, most preferably 2-5 wt.-%, based on the total weight of the gummy product, of at least one gelling agent.

The gummy product can comprise of any commonly known ingredient (auxiliary agent) used for such formulations, such as sweeteners (such as i.e. sucrose and/or sugar alcohol and/or other types of sugars (also known as dextrose, corn syrup, high fructose corn syrup, etc.)), flavors (natural ones as well as artificial ones), colors (natural ones as well as artificial ones), antioxidants (i.e. green tea extract, ascorbic acid, rosemary extract, and tocopherols), and pH buffer.

Such ingredients (auxiliary agents) are usually present in an amount of 0.1-70 wt.-%, (preferably 1-70 wt.-%), based on the total weight of the gummy product. The optional auxiliary agent may comprise the balance of the total weight of the gummy product after accounting for the amount of oily ingredient, emulsifier, water, and gelling agent present in the gummy product. For example, in an embodiment, the amount of oily ingredient, emulsifier, water, and gelling agent are in total present at an amount of 39 wt.-% and the auxiliary agents is present at a total amount of 61 wt.-%.

Preferably, the content of at least one antioxidant in the gummy product is 0.1-2 wt.-%, (more preferably 0.2-1 wt.-%), based on the total weight of the gummy product.

Preferably, the content of at least one flavor in the gummy product is 0.1-5 wt.-%, (more preferably 0.2-4 wt.-%), based on the total weight of the gummy product.

Preferably, the content of at least one color in the gummy product is 0.05-5 wt.-%, (more preferably 0.2-2 wt.-%), based on the total weight of the gummy product.

Furthermore, the gummy product can also comprise further active ingredients (next to the oily ingredient).

Preferred active ingredients for the embodiment of the present invention may include wa-ter-soluble vitamins, HMOs, minerals, nutraceuticals, glycosaminoglycan or its active members, prebiotics, probiotics, amino acids, and any other dietary ingredient or combi-nation thereof that are useful in human or animal nutrition.
Such additional active ingredients may be present in amount of up to 25 wt.-%, based on the total weight of the gummy product. Preferably up to 10 wt.-%, based on the total weight of the gummy product.

The size and the shape of the gummy product can vary.

A usual form of the gummy product is a spere-like shape, an egg-like shape, or a form of an animal (such as bears, cats, dogs, turtles, snakes, etc.)
A usual diameter of the longest dimension of the gummy product is in the range of 0.5 cm-5 cm.

A usual weight of the gummy product is in the range of 0.5 to 10 g.

The color of the gummy product is usually chosen in such a way that it matches the flavor of the gummy product. (yellow=citron, green=algal).

A general way to produce the gummy product according to the present invention is the following:
1) Blend gelling agent, buffering agent, and about 10% of solid sweetener. Add the blend to water (gelling agent:water weight ratio=1:10) and hydrate solution at 80-85° C. for 30 min.
2) Add syrup, and about 90% of solid sweetener to a vessel and cook to 90-95° C.
3) Add hydrated gelling agent slurry (from Step 1) into the syrup/sweetener slurry and continue heating until Brix reaches 78% (Range 70%-85%).
4) Prepare emulsifier/oil/flavor (/antioxidant blend for Omega-3 gummies) emulsion. Dry mix emulsifier(s) (and antioxidant blend for Omega-3 gummies), then mix into water (powder: water weight ratio=1:2) using an overhead mixer at 200-500 rpm until uniform. Blend the oil and flavor and mix into the emulsifier solution with the overhead mixer for 5 minutes at 200-500 rpm. Shear mix the mixture for 2 minutes at 6000 rpm with high shear mixer.
5) Add the emulsion from step 4 and color into the mixture from step 3, mix well until homogeneous, and continue heating until Brix reaches 78% (Range 70%-85%).
6) Prepare citric acid solution (citric acid: water weight ratio=1:1), add into mixture from step 5, and mix well. Check Brix (Target 78%, range 70%-85%).
7) Deposit into silicone molds. Remove gummies from mold and polish with car-nauba wax after 24 hours.

The invention is illustrated by the following Examples. All temperatures are given in ° C. and all parts and percentages are related to the weight.

### EXAMPLES

Phase separation is an indication of a poor-quality gummy. Phase separation is associated with undesirable stability, taste, smell, appearance, and other undesirable characteristics of a gummy. In the following examples it is demonstrated that gummies in accordance with the invention do not show phase separation relative to the comparative examples.

### Example 1: Pectin-Based High CBD Oil Gummy, 16 mg CBD/1 Gummy (4.7 g)

| Ingredient | % |
|---|---|
| CBD Oil (1.67% concentration) | 20.00 |
| Gum Arabic | 1.50 |
| Starch | 2.00 |
| Pectin APC 165B | 2.10 |
| Water | 11.00 |
| Xylitol | 22.00 |
| Allulose Syrup | 21.21 |
| Maltitol Syrup | 18.54 |
| Citric Acid | 0.80 |
| Color | 0.25 |
| Orange Flavor | 0.40 |
| Sodium Citrate | 0.20 |
| Total | 100.00 |

Total weight of the gummy product: 4.7 g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare gum Arabic/starch/CBD oil/flavor emulsion. Dry mix gum Arabic and starch, then mix into water (powder: water weight ratio=1:2) using an overhead mixer at 200-500 rpm until uniform. Blend the CBD oil and flavor and mix into the gum Arabic/starch solution with the overhead mixer for 5 minutes at 200-500 rpm. Shear mix the gum Arabic/starch/CBD oil/flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 2: Pectin-Based High Vitamin Oil Gummy, Vitamin Oil (Vitamin A: 846 Mcg RAE, Vitamin D: 375 Mcg, Vitamin E: 282 mg, Vitamin K1: 677 Mcg)/1 Gummy (4.7 g)

| Ingredient | % |
|---|---|
| Vitamin Premix Oil (see Table 1 | 20.00 |
| below for ingredient breakdown) | |
| Gum Arabic | 1.50 |
| Starch | 2.00 |
| Pectin APC 165B | 2.10 |
| Water | 11.00 |
| Xylitol | 22.00 |
| Allulose Syrup | 21.21 |
| Maltitol Syrup | 18.54 |
| Citric Acid | 0.80 |
| Color | 0.25 |
| Orange Flavor | 0.40 |
| Sodium Citrate | 0.20 |
| Total | 100.00 |

Total weight of the gummy product: 4.7 g

TABLE 1
Ingredients of the Vitamin Premix Oil
Ingredients of the Vitamin Premix Oil

| | |
|---|---|
| Vitamin A | 3.21% |
| Vitamin D | 6.42% |
| Vitamin E | 69.10% |
| Vitamin K1 | 0.16% |
| Safflower Oil | 21.11% |

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare gum Arabic/starch/vitamin oil/flavor emulsion. Dry mix gum Arabic and starch, then mix into water (powder: water weight ratio=1:2) using an overhead mixer at 200-500 rpm until uniform. Blend the vitamin oil and flavor and mix into the gum Arabic/starch solution with the overhead mixer for 5 minutes at 200-500 rpm. Shear mix the gum Arabic/starch/vitamin oil/flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 3: Pectin-Based High Mixed Tocopherols Oil Gummy, 893 mg Mixed Tocopherols/1 Gummy (4.7 g)

| Ingredient | % |
|---|---|
| Mixed Tocopherols 95% Oil | 20.00 |
| Gum Arabic | 1.50 |
| Starch | 2.00 |
| Pectin APC 165B | 2.10 |
| Water | 11.00 |
| Xylitol | 22.00 |
| Allulose Syrup | 21.21 |
| Maltitol Syrup | 18.54 |
| Citric Acid | 0.80 |
| Color | 0.25 |
| Orange Flavor | 0.40 |
| Sodium Citrate | 0.20 |
| Total | 100.00 |

Total weight of the gummy product: 4.7 g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare gum Arabic/starch/mixed tocopherols oil/flavor emulsion. Dry mix gum Arabic and starch, then mix into water (powder: water weight ratio=1:2) using an overhead mixer at 200-500 rpm until uniform. Blend the mixed tocopherols oil and flavor and mix into the gum Arabic/starch solution with the overhead mixer for 5 minutes at 200-500 rpm. Shear mix the gum Arabic/starch/mixed tocopherols oil/flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 4: Pectin-Based High Omega-3 Gummy, 470 mg DHA+EPA/1 Gummy (4.7 g)

| Ingredient | % |
|---|---|
| Life's Omega 60 | 20.20 |
| Quillaja Extract | 10.00 |
| Pectin APC 165B | 2.10 |
| Water | 8.00 |
| Xylitol | 25.00 |
| Allulose Syrup | 10.36 |
| Maltitol Syrup | 20.68 |
| Citric Acid | 0.80 |
| Antioxidant Blend | 0.40 |
| Reb M Stevia | 0.01 |
| Color | 0.25 |
| Natural Masking Flavor | 1.00 |
| Mixed Berry Lemonade Flavor | 1.00 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare quillaja extract/antioxidant blend/Reb M/algal oil/flavor/masking flavor emulsion. Mix antioxidant blend and Reb M with quillaja extract, using an overhead mixer at 200-500 rpm until uniform. Blend the algal oil, flavor, and masking flavor, and mix into the quillaja extract solution with the overhead mixer for 5 minutes at 200-500 rpm. Shear mix the quillaja extract/antioxidant blend/Reb M/algal oil/flavor/masking flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 5: Pectin-Based High Omega-3 Gummy, 342 mg DHA+EPA/1 Gummy (4.7 g)

| Ingredient | % |
|---|---|
| Life's Omega 45 | 19.27 |
| Modified Food Starch | 2.00 |
| Pectin APC 165B | 2.10 |
| Water | 11.00 |
| Xylitol | 25.00 |
| Allulose Syrup | 21.21 |
| Maltitol Syrup | 15.36 |
| Citric Acid | 0.80 |
| Antioxidant Blend | 0.80 |
| Reb M Stevia | 0.01 |
| Color | 0.25 |
| Natural Masking Flavor | 1.00 |
| Mixed Berry Lemonade Flavor | 1.00 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare modified food starch/antioxidant blend/Reb M/algal oil/flavor/masking fla-vor emulsion. Dry mix modified food starch, antioxidant blend, and Reb M, then mix into water (powder: water weight ratio=1:2) using an overhead mixer at 200-500 rpm until uniform. Blend the algal oil, flavor, and masking flavor, and mix into the modified food starch solution with the overhead mixer for 5 minutes at 200-500 rpm. Shear mix the modified food starch/antioxidant blend/Reb M/algal oil/flavor/masking flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 6: Pectin-Based High Omega-3 Gummy, 470 mg DHA+EPA/1 Gummy (4.7 g)

| Ingredient | % |
|---|---|
| Life's Omega 60 | 20.20 |
| Modified Food Starch | 2.50 |
| Pectin APC 165B | 2.10 |
| Water | 11.00 |
| Xylitol | 25.00 |
| Allulose Syrup | 21.21 |
| Maltitol Syrup | 13.73 |
| Citric Acid | 0.80 |
| Antioxidant Blend | 0.80 |
| Reb M Stevia | 0.01 |
| Color | 0.25 |
| Natural Masking Flavor | 1.10 |
| Mixed Berry Lemonade Flavor | 1.10 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare modified food starch/antioxidant blend/Reb M/algal oil/flavor/masking fla-vor emulsion. Dry mix modified food starch, antioxidant blend, and Reb M, then mix into water (powder: water weight ratio=1:2) using an overhead mixer at 200-500 rpm until uniform. Blend the algal oil, flavor, and masking flavor, and mix into the modified food starch solution with the overhead mixer for 5 minutes at 200-500 rpm. Shear mix the modified food starch/antioxidant blend/Reb M/algal oil/flavor/masking flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 7: Pectin-Based High Omega-3 Gummy, 411 mg DHA+EPA/1 Gummy (4.7 g)

| Ingredient | % |
|---|---|
| Life's Omega 45 | 22.53 |
| Modified Food Starch | 2.00 |
| Pectin APC 165B | 2.10 |
| Water | 11.00 |
| Xylitol | 25.00 |
| Allulose Syrup | 21.21 |
| Maltitol Syrup | 12.10 |
| Citric Acid | 0.80 |
| Antioxidant Blend | 0.80 |
| Reb M Stevia | 0.01 |
| Color | 0.25 |
| Natural Masking Flavor | 1.00 |
| Mixed Berry Lemonade Flavor | 1.00 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare modified food starch/antioxidant blend/Reb M/algal oil/flavor/masking fla-vor emulsion. Dry mix modified food starch, antioxidant blend, and Reb M, then mix into water (powder: water weight ratio=1:2) using an overhead mixer at 200-500 rpm until uniform. Blend the algal oil, flavor, and masking flavor, and mix into the modified food starch solution with the overhead mixer for 5 minutes at 200-500 rpm. Shear mix the modified food starch/antioxidant blend/Reb M/algal oil/flavor/masking flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 8: Pectin-Based High Omega-3 Gummy, 411 mg DHA+EPA/1 Gummy (4.7 g)

| Ingredient | % |
|---|---|
| Life's Omega 45 | 22.53 |
| Gum Arabic | 3.50 |
| Pectin APC 165B | 2.10 |
| Water | 11.00 |
| Xylitol | 25.00 |
| Allulose Syrup | 21.21 |
| Maltitol Syrup | 10.40 |
| Citric Acid | 0.80 |
| Antioxidant Blend | 0.80 |
| Reb M Stevia | 0.01 |
| Color | 0.25 |
| Natural Masking Flavor | 1.10 |
| Mixed Berry Lemonade Flavor | 1.10 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare gum Arabic/antioxidant blend/Reb M/algal oil/flavor/masking flavor emulsion. Dry mix gum Arabic, antioxidant blend, and Reb M, then mix into water (powder: water weight ratio=1:2) using an overhead mixer at 200-500 rpm until uniform. Blend the algal oil, flavor, and masking flavor, and mix into the gum Arabic solution with the overhead mixer for 5 minutes at 200-500 rpm. Shear mix the gum Arabic/antioxidant blend/Reb M/algal oil/flavor/masking flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 9: Pectin-Based High Omega-3 Gummy, 470 mg DHA+EPA/1 Gummy (4.7 g)

| Ingredient | % |
|---|---|
| Life's Omega 60 | 20.20 |
| Gum Arabic | 2.50 |
| Pectin APC 165B | 2.10 |
| Water | 11.00 |
| Xylitol | 25.00 |
| Allulose Syrup | 21.21 |
| Maltitol Syrup | 13.73 |
| Citric Acid | 0.80 |
| Antioxidant Blend | 0.80 |
| Reb M Stevia | 0.01 |
| Color | 0.25 |
| Natural Masking Flavor | 1.10 |
| Mixed Berry Lemonade Flavor | 1.10 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare gum Arabic/antioxidant blend/Reb M/algal oil/flavor/masking flavor emulsion. Dry mix gum Arabic, antioxidant blend, and Reb M, then mix into water (powder: water weight ratio=1:2) using an overhead mixer at 200-500 rpm until uniform. Blend the algal oil, flavor, and masking flavor, and mix into the gum Arabic solution with the overhead mixer for 5 minutes at 200-500 rpm. Shear mix the gum Arabic/antioxidant blend/Reb M/algal oil/flavor/masking flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 10: Pectin-Based High Omega-3 Gummy, 411 mg DHA+EPA/1 Gummy (4.7 g)

| Ingredient | % |
|---|---|
| Life's Omega 45 | 22.53 |
| Gum Arabic | 1.50 |
| Starch | 2.00 |
| Pectin APC 165B | 2.10 |
| Water | 11.00 |
| Xylitol | 25.00 |
| Allulose Syrup | 21.21 |
| Maltitol Syrup | 10.40 |
| Citric Acid | 0.80 |
| Antioxidant Blend | 0.80 |
| Reb M Stevia | 0.01 |
| Color | 0.25 |
| Natural Masking Flavor | 1.10 |
| Mixed Berry Lemonade Flavor | 1.10 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare gum Arabic/starch/antioxidant blend/Reb M/algal oil/flavor/masking fla-vor emulsion. Dry mix gum Arabic, starch, antioxidant blend, and Reb M, then mix into water (powder: water weight ratio=1:2) using an overhead mixer at 200-500 rpm until uniform. Blend the algal oil, flavor, and masking flavor, and mix into the gum Arabic/starch solution with the overhead mixer for 5 minutes at 200-500 rpm. Shear mix the gum Arabic/starch/antioxidant blend/Reb M/algal oil/fla-vor/masking flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 11: Pectin-Based High Omega-3 Gummy, 470 mg DHA+EPA/1 Gummy (4.7 g)

| Ingredient | % |
|---|---|
| Life's Omega 60 | 20.20 |
| Gum Arabic | 1.50 |
| Starch | 2.00 |
| Pectin APC 165B | 2.10 |
| Water | 11.00 |
| Xylitol | 25.00 |
| Allulose Syrup | 21.21 |
| Maltitol Syrup | 12.73 |
| Citric Acid | 0.80 |
| Antioxidant Blend | 0.80 |
| Reb M Stevia | 0.01 |
| Color | 0.25 |
| Natural Masking Flavor | 1.10 |
| Mixed Berry Lemonade Flavor | 1.10 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Prepare gum Arabic/starch/antioxidant blend/Reb M/algal oil/flavor/masking fla-vor emulsion. Dry mix gum Arabic, starch, antioxidant blend, and Reb M, then mix into water (powder: water weight ratio=1:2) using an overhead mixer at 200-500 rpm until uniform. Blend the algal oil, flavor, and masking flavor, and mix into the gum Arabic/starch solution with the overhead mixer for 5 minutes at 200-500 rpm. Shear mix the gum Arabic/starch/antioxidant blend/Reb M/algal oil/fla-vor/masking flavor mixture for 2 minutes at 6000 rpm with Silverson high speed mixer.
5. Add the emulsion from step 4 and the color into the mixture from step 3, mix well until homogeneous, and continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

### Example 12 (Comparison Example): Pectin-Based High CBD Oil Gummy, 12 mg CBD/1 Gummy (4.7 g)

| Ingredient | % |
|---|---|
| CBD Oil (1.67% concentration) | 15.00 |
| Pectin APC 165B | 2.10 |
| Water | 11.00 |
| Xylitol | 22.00 |
| Allulose Syrup | 21.21 |
| Maltitol Syrup | 27.04 |
| Citric Acid | 0.80 |
| Color | 0.25 |
| Orange Flavor | 0.40 |
| Sodium Citrate | 0.20 |
| Total | 100.00 |

Total weight of the gummy product: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Add flavor, color, and CBD oil into the solution from step 3 and mix well until homogenous. Continue heating until Brix reaches 78% (Range 70%-85%).
5. Prepare citric acid solution (citric acid: water weight ratio=1:1), add into mixture from step 5, and mix well. Check Brix (Target 78%, range 70%-85%).
6. Deposit into silicone molds. Remove gummies from mold and polish with car-nauba wax after 24 hours.
   Phase separation between the oil and the matrix was observed. No stable gummy product has been obtained.

### Example 13 (Comparison Example): Pectin-Based High Vitamin Oil Gummy, Vitamin Oil (Vitamin A: 635 Mcg RAE, Vitamin D: 282 Mcg, Vitamin E: 212 mg, Vitamin K1: 508 Mcg)/1 Gummy (4.7 g)

| Ingredient | % |
|---|---|
| Vitamin Premix Oil (see Table 2 below for ingredient | 15.00 |
| breakdown) | |
| Pectin APC 165B | 2.10 |
| Water | 11.00 |
| Xylitol | 22.00 |
| Allulose Syrup | 21.21 |
| Maltitol Syrup | 27.04 |
| Citric Acid | 0.80 |
| Color | 0.25 |
| Orange Flavor | 0.40 |
| Sodium Citrate | 0.20 |
| Total | 100.00 |

Total weight per gummy: 4.7g

TABLE 2
Ingredients of Vitamin Premix Oil

| | |
|---|---|
| Vitamin A | 3.21% |
| Vitamin D | 6.42% |
| Vitamin E | 69.10% |
| Vitamin K1 | 0.16% |
| Safflower Oil | 21.11% |

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Add flavor, color, and vitamin premix oil into the solution from step 3 and mix well until homogenous. Continue heating until Brix reaches 78% (Range 70%-85%).
5. Prepare citric acid solution (citric acid: water weight ratio=1:1), add into mixture from step 5, and mix well. Check Brix (Target 78%, range 70%-85%).
6. Deposit into silicone molds. Remove gummies from mold and polish with car-nauba wax after 24 hours.

Phase separation between the oil and the matrix was observed. No stable gummy product has been obtained.

### Example 14 (Comparison Example): Pectin-Based High Mixed Tocopherols Oil Gummy, 670 mg Mixed Tocopherols/1 Gummy (4.7 g)

| Ingredient | % |
|---|---|
| Mixed Tocopherols 95% Oil | 15.00 |
| Pectin APC 165B | 2.10 |
| Water | 11.00 |
| Xylitol | 22.00 |
| Allulose Syrup | 21.21 |
| Maltitol Syrup | 27.04 |
| Citric Acid | 0.80 |
| Color | 0.25 |
| Orange Flavor | 0.40 |
| Sodium Citrate | 0.20 |
| Total | 100.00 |

Total weight per gummy: 4.7g

### Procedure:

1. Blend pectin, sodium citrate, and about 10% of xylitol. Add the blend to water (pectin: water weight ratio=1:10) and hydrate pectin solution at 80-85° C. for 30 min.
2. Add allulose syrup, maltitol syrup, and about 90% of xylitol to a vessel and cook to 90-95° C.
3. Add hydrated pectin slurry (from Step 1) into the syrup/xylitol slurry and continue heating until Brix reaches 78% (Range 70%-85%).
4. Add flavor, color, and mixed tocopherols oil into the solution from step 3 and mix well until homogenous. Continue heating until Brix reaches 78% (Range 70%-85%).
5. Prepare citric acid solution (citric acid: water weight ratio=1:1), add into mixture from step 5, and mix well. Check Brix (Target 78%, range 70%-85%).
6. Deposit into silicone molds. Remove gummies from mold and polish with car-nauba wax after 24 hours.

Phase separation between the oil and the matrix was observed. No stable gummy product has been obtained.

### Example 15 (Comparison Example): Pectin-Based High Omega-3 Gummy, 705 mg DHA+EPA/1 Gummy (4.7 g)

| Ingredient | % |
|---|---|
| Life's Omega 60 | 28.91 |
| Pectin APC 165B | 13.00 |
| Water | 14.00 |
| Sugar | 29.00 |
| Corn Syrup | 8.89 |
| Citric Acid | 1.60 |
| Ascorbic Acid | 0.09 |
| Dry Mixed Tocopherols | 0.10 |
| Rosemary Extract | 0.09 |
| Green Tea Extract | 0.12 |
| Color | 2.00 |
| Natural Masking Flavor | 0.90 |
| Mixed Berry Lemonade Flavor | 0.90 |
| Sodium Citrate | 0.40 |
| Total | 100.00 |

Total weight per gummy: 4.7g

### Procedure:

1. Blend pectin, sodium citrate, and about 10% of sugar. Add the blend to water (pectin: water weight ratio=1:10) and hydrate pectin solution at 80-85° C. for 30 min.
2. Add corn syrup, and about 90% of sugar to a vessel and cook to 90-95° C.
3. Add hydrated pectin slurry (from Step 1) into the syrup/sugar slurry and continue heating until Brix reaches 78% (Range 70%-85%).
4. Prepare antioxidant blend slurry by adding the antioxidant blend into water and mixing (antioxidant blend: water weight ratio=1:1). Add the antioxidant slurry into the mixture, mix well until homogeneous, and continue heating until Brix
5. Add flavors, color, and Life's Omega 60 oil into the solution from step 3 and mix will until homogenous. Continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

Phase separation between the oil and the matrix was observed. No stable gummy product has been obtained.

### Example 16 (Comparison Example): Pectin-Based High Omega-3 Gummy, 470 mg DHA+EPA/1 Gummy (4.7 g)

| Ingredient | % |
|---|---|
| Life's Omega 60 | 20.20 |
| Pectin APC 165B | 2.10 |
| Water | 11.00 |
| Sugar | 33.00 |
| Corn Syrup | 28.18 |
| Citric Acid | 1.00 |
| Ascorbic Acid | 0.09 |
| Dry Mixed Tocopherols | 0.10 |
| Rosemary Extract | 0.09 |
| Green Tea Extract | 0.12 |
| Color | 2.00 |
| Natural Masking Flavor | 0.90 |
| Mixed Berry Lemonade Flavor | 0.90 |
| Sodium Citrate | 0.32 |
| Total | 100.00 |

Total weight per gummy: 4.7g

### Procedure:

1. Blend pectin, sodium citrate, and about 10% of sugar. Add the blend to water (pectin: water weight ratio=1:10) and hydrate pectin solution at 80-85° C. for 30 min.
2. Add corn syrup, and about 90% of sugar to a vessel and cook to 90-95° C.
3. Add hydrated pectin slurry (from Step 1) into the syrup/sugar slurry and continue heating until Brix reaches 78% (Range 70%-85%).
4. Prepare antioxidant blend slurry by adding the antioxidant blend into water and mixing (antioxidant blend: water weight ratio=1:1). Add the antioxidant slurry into the mixture, mix well until homogeneous, and continue heating until Brix reaches 78% (Range 70%-85%).
5. Add flavors, color, and Life's Omega 60 oil into the solution from step 3 and mix will until homogenous. Continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

Phase separation between the oil and the matrix was observed. No stable gummy product has been obtained.

### Example 17 (Comparison Example): Pectin-Based High Omega-3 Gummy, 235 mg DHA+EPA/1 Gummy (4.7 g)

| Ingredient | % |
|---|---|
| Life's Omega 60 | 15.48 |
| Pectin APC 165B | 2.10 |
| Water | 11.00 |
| Sugar | 33.00 |
| Corn Syrup | 32.90 |
| Citric Acid | 1.00 |
| Ascorbic Acid | 0.09 |
| Dry Mixed Tocopherols | 0.10 |
| Rosemary Extract | 0.09 |
| Green Tea Extract | 0.12 |
| Color | 2.00 |
| Natural Masking Flavor | 0.90 |
| Mixed Berry Lemonade Flavor | 0.90 |
| Sodium Citrate | 0.32 |
| Total | 100.00 |

Total weight per gummy: 4.7g

### Procedure:

1. Blend pectin, sodium citrate, and about 10% of sugar. Add the blend to water (pectin: water weight ratio=1:10) and hydrate pectin solution at 80-85° C. for 30 min.
2. Add corn syrup, and about 90% of sugar to a vessel and cook to 90-95° C.
3. Add hydrated pectin slurry (from Step 1) into the syrup/sugar slurry and continue heating until Brix reaches 78% (Range 70%-85%).
4. Prepare antioxidant blend slurry by adding the antioxidant blend into water and mixing (antioxidant blend: water weight ratio=1:1). Add the antioxidant slurry into the mixture, mix well until homogeneous, and continue heating until Brix
5. Add flavors, color, and Life's Omega 60 oil into the solution from step 3 and mix will until homogenous. Continue heating until Brix reaches 78% (Range 70%-85%).
6. Prepare citric acid solution (citric acid: water weight ratio=1:1), add into mixture from step 5, and mix well. Check Brix (Target 78%, range 70%-85%).
7. Deposit into silicone molds. Remove gummies from mold and polish with car-nauba wax after 24 hours.

Phase separation between the oil and the matrix was observed. No stable gummy product has been obtained.

1. A gummy product comprising:
10-30 wt.-%, based on the weight of the gummy product, of at least one oily ingredient, and
0.5-20 wt.-%, based on the total weight of the gummy product, of at least one emulsifier, and
5-35 wt.-%, based on the total weight of the gummy product, of water, and
1-15 wt.-%, based on the total weight of the gummy product, of at least one gelling agent, wherein the gelling agent comprises less than 4 wt.-% gelatin, and
0.1-70 wt.-%, based on the total weight of the gummy product, of at least one auxiliary agent, and
wherein the at least one oily ingredient is distributed homogenously in the gummy product.

2. The gummy product according to claim 1, wherein at least one oily ingredient is chosen from the group consisting of PUFAs, fat-soluble vitamins, carotenoids, CBD oils, and mixtures thereof.

3. The gummy product according to any of the preceding claims, wherein at least one emulsifier is chosen from the group consisting of modified food starch, quillaja extract, gum Arabic, modified gum Acacia, polysorbates, polyglycerol esters of fatty acids, lecithin, sucrose esters, sucroglycerides, lignosulfonates, and mixtures thereof.

4. A gummy product comprising:
15-25 wt.-%, based on the weight of the gummy product, of oily ingredients se-lected from the group consisting of PUFAs, fat-soluble vitamins, carotenoids, CBD oils, and mixtures thereof,
1-8 wt.-%, based on the total weight of the gummy product, of at least one emulsifier, wherein emulsifier comprises modified food starch or gum Arabic,
10-30 wt.-%, based on the total weight of the gummy product, of water, and
2-8 wt.-%, based on the total weight of the gummy product, of at least one gelling agent, and
0.1-70 wt.-%, based on the total weight of the gummy product, of at least one auxiliary agent, and
wherein the at least one oily ingredient is distributed homogenously in the gummy product.

5. The gummy product according to any of the preceding claims, wherein at least one gelling agent is chosen from the group consisting of gelatin (from any source), pectin (from any sources), starches (from any sources), carrageenan, gellan gum, agar, other hydrocolloids (from any sources), and mixtures thereof.

6. The gummy product according to any of the preceding claims, wherein the gummy product comprises at least one auxiliary agent chosen from the group consisting of sweeteners (sucrose, glucose syrup, allulose, sucralose, stevia, Monk fruit powder, sugar alcohols such as xylitol, maltitol, erythritol), flavors (natural ones as well as artificial ones), colors (natural ones as well as artificial ones), antioxidants (i.e. green tea extract, ascorbic acid, rosemary extract, and tocopherols), pH buffer (citric acid, Sodium citrate, malic acid, lactic acid, Sodium lactate, tartaric acid, fumaric acid), and mixtures thereof.

7. The gummy product according to any of the preceding claims, wherein the gummy product comprises at least one additional active ingredient (next to the at least one oily ingredient) chosen from the group consisting of water-soluble vitamins, HMOs, minerals, nutraceuticals, glycosaminoglycan or its active members, prebiotics, probiotics, amino acids, and mixtures thereof.

8. The product according to claim 7, wherein the content of the least one additional active ingredient in the gummy product is up to 25 wt.-%, based on the total weight of the gummy product.

9. The gummy product according to any of the preceding claims, wherein the content of the at least one oily ingredient in the gummy product is 18-25 wt.-%, based on the total weight of the gummy product.

10. The product according to any of the preceding claims, wherein the content of the at least one emulsifier in the gummy product is 1-8 wt.-%, based on the total weight of the gummy product.

11. The product according to any of the preceding claims, wherein the content of water in the gummy product is 10-28 wt.-%, based on the total weight of the gummy product.

12. The product according to any of the preceding claims, wherein the content of the at least one gelling agent in the gummy product is 2-6 wt.-%, based on the total weight of the gummy product, more preferably 2-5 wt.-%.

13. The product according to any of the preceding claims, wherein the gelling agent is devoid of gelatin.

14. The product according to any one of the preceding claims, wherein the gelling agent consists of a pectin, a starch, or a mixture thereof.

15. A process for producing gummy products according to any of claims 1-12, comprising the following steps:
1) Blend gelling agent, buffering agent, and about 10% of solid sweetener. Add the blend to water (gelling agent: water weight ratio=1:10) and hydrate solution at 80-85° C. for 30 min.
2) Add syrup, and about 90% of solid sweetener to a vessel and cook to 90-95° C.
3) Add hydrated gelling agent slurry (from Step 1) into the syrup/sweetener slurry and continue heating until Brix reaches 78% (Range 70%-85%).
4) Prepare emulsifier/oil/flavor (/antioxidant blend for Omega-3 gummies) emulsion. Dry mix emulsifier(s) (and antioxidant blend for Omega-3 gummies), then mix into water (powder: water weight ratio=1:2) using an overhead mixer at 200-500 rpm until uniform. Blend the oil and flavor and mix into the emulsifier solution with the overhead mixer for 5 minutes at 200-500 rpm. Shear mix the mixture for 2 minutes at 6000 rpm with high shear mixer.
5) Add the emulsion from step 4 and color into the mixture from step 3, mix well until homogeneous, and continue heating until Brix reaches 78% (Range 70%-85%).
6) Prepare citric acid solution (citric acid: water weight ratio=1:1), add into mixture from step 5, and mix well. Check Brix (Target 78%, range 70%-85%).
7) Deposit into silicone molds. Remove gummies from mold and polish with car-nauba wax after 24 hours.
